(19) United States
(12) Patent Application Publication
(10) Pub. No.: US 2019/0343738 A1
(43) Pub. Date: Nov. 14, 2019

(54) Oral Care Compositions Comprising Zinc Compounds and Peroxide Compounds

(71) Applicant: Colgate-Palmolive Company, New York, NY (US)

(72) Inventors: Kyle ROBBINS, Toms River, NJ (US); Jason NESTA, Cedar Knolls, NJ (US); Suman CHOPRA, Monroe, NJ (US)

(73) Assignee: Colgate-Palmolive Company, New York, NY (US)

(21) Appl. No.: 16/520,768

(22) Filed: Jul. 24, 2019

Related U.S. Application Data
(63) Continuation of application No. 15/520,224, filed on Apr. 19, 2017.
(60) Provisional application No. 62/067,950, filed on Oct. 23, 2014.

(51) Int. Cl. A61K 8/22; A61Q 11/00; A61K 8/27

(52) U.S. Cl. CPC A61K 8/22; A61Q 11/00; A61K 2800/31; A61K 2800/40; A61K 8/27

(57) ABSTRACT

The present invention provides an oral care composition comprising a peroxide compound and a zinc compound, wherein the weight ratio of the peroxide compound to the zinc compound is from 1:10 to 5:1.

### BACKGROUND

Peroxide compounds are added to oral care compositions such as dentifrices, mouthrinses, strips and gels to whiten teeth through the bleaching of stains. However, many countries regulate the level of hydrogen peroxide allowable in these products to a maximum of 0.1 weight %. It would be desirable to improve the whitening benefits of these products.

### BRIEF SUMMARY

In a first aspect, the present invention provides an oral care composition comprising a peroxide compound and a zinc compound, wherein the weight ratio of the peroxide compound to the zinc compound is from 1:10 to 5:1.

Optionally, the weight ratio of the peroxide compound to the zinc salt is from 1:8 to 4:1, optionally from 1:5 to 3:1, optionally from 1:3 to 2:1, optionally from 1:2 to 1:1, further optionally about 1:1.

Optionally, the zinc compound is selected from zinc oxide, zinc citrate, zinc chloride, zinc acetate, zinc lactate, zinc salicylate, zinc sulfate, zinc phosphate, zinc tartrate and zinc nitrate. Further optionally, the zinc compound is zinc oxide.

Optionally, the zinc compound is present in a concentration of from 0.1 to 5 weight %, optionally from 0.75 to 1.5 weight %, based on the weight of the composition.

Optionally, the peroxide compound is hydrogen peroxide, a peroxide of an alkali metal, a peroxide of an alkaline earth metal, an organic peroxy compound, or a peroxy acid. Further optionally, the peroxide compound is hydrogen peroxide. Still further optionally, the hydrogen peroxide is present as a complex with a polymer. Yet further optionally, the polymer is polyvinylpyrrolidone.

Optionally, the peroxide compound is present in a concentration of from 0.01 to 0.5 weight %, optionally from 0.05 to 0.1 weight %, based on the total weight of the composition.

Optionally, the composition is anhydrous.

Optionally, the composition is a toothpaste, a liquid, a gel, or a whitening strip.

In a second aspect, the present invention provides a method of increasing the whitening efficacy of an oral care composition which comprises a peroxide compound, the method comprising formulating the composition so as to further comprise a zinc compound, wherein the weight ratio of the peroxide compound to the zinc compound in the composition is from 1:10 to 5:1.

Optionally, the weight ratio of the peroxide compound to the zinc salt is from 1:8 to 4:1, optionally from 1:5 to 3:1, optionally from 1:3 to 2:1, optionally from 1:2 to 1:1, further optionally about 1:1.

Optionally, the zinc compound is selected from zinc oxide, zinc citrate, zinc chloride, zinc acetate, zinc lactate, zinc salicylate, zinc sulfate, zinc phosphate, zinc tartrate and zinc nitrate. Further optionally, the zinc compound is zinc oxide.

Optionally, the zinc compound is present in the composition in a concentration of from 0.1 to 5 weight %, optionally from 0.75 to 1.5 weight %, based on the weight of the composition.

Optionally, the peroxide compound is hydrogen peroxide, a peroxide of an alkali metal, a peroxide of an alkaline earth metal, an organic peroxy compound, or a peroxy acid. Further optionally, the peroxide compound is hydrogen peroxide. Still further optionally, the hydrogen peroxide is present as a complex with a polymer. Yet further optionally, the polymer is polyvinylpyrrolidone.

Optionally, the peroxide compound is present in the composition in a concentration of from 0.01 to 0.5 weight %, optionally from 0.05 to 0.1 weight %, based on the total weight of the composition.

Optionally, the composition is anhydrous.

Optionally, the composition is a toothpaste, a liquid, a gel, or a whitening strip.

In a third aspect, the present invention provides the use, in an oral care composition which comprises a peroxide compound, of a zinc compound to increase the whitening efficacy of the composition, wherein the weight ratio of the peroxide compound to the zinc compound in the composition is from 1:10 to 5:1.

Optionally, the weight ratio of the peroxide compound to the zinc salt is from 1:8 to 4:1, optionally from 1:5 to 3:1, optionally from 1:3 to 2:1, optionally from 1:2 to 1:1, further optionally about 1:1.

Optionally, the zinc compound is selected from zinc oxide, zinc citrate, zinc chloride, zinc acetate, zinc lactate, zinc salicylate, zinc sulfate, zinc phosphate, zinc tartrate and zinc nitrate. Further optionally, the zinc compound is zinc oxide.

Optionally, the zinc compound is present in the composition in a concentration of from 0.1 to 5 weight %, optionally from 0.75 to 1.5 weight %, based on the weight of the composition.

Optionally, the peroxide compound is hydrogen peroxide, a peroxide of an alkali metal, a peroxide of an alkaline earth metal, an organic peroxy compound, or a peroxy acid. Further optionally, the peroxide compound is hydrogen peroxide. Still further optionally, the hydrogen peroxide is present as a complex with a polymer. Yet further optionally, the polymer is polyvinylpyrrolidone.

Optionally, the peroxide compound is present in the composition in a concentration of from 0.01 to 0.5 weight %, optionally from 0.05 to 0.1 weight %, based on the total weight of the composition.

Optionally, the composition is anhydrous.

Optionally, the composition is a toothpaste, a liquid, a gel, or a whitening strip.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

All ratios expressed herein should be understood to refer to ratios by weight, unless otherwise indicated.

As discussed above, it would be desirable to provide oral care compositions in which the whitening efficacy is improved, even when relatively low levels of peroxide compound are included.

Therefore, in one aspect of the present invention, there is provided an oral care composition comprising a peroxide compound and a zinc compound, wherein the weight ratio of the peroxide compound to the zinc compound is from 1:10 to 5:1.

The present inventors have surprisingly found that the inclusion of a zinc compound in an oral care composition comprising a peroxide compound, at the above ratio of zinc compound to peroxide compound, results in a significant improvement in the whitening efficacy of the composition. This result is particularly surprising, and cannot be explained by any antibacterial activity of the zinc compound or by any change in pH caused by the zinc compound (as discussed in the Examples, below). This discovery allows for use of a lower amount of peroxide compound while maintaining the efficacy of a higher amount of peroxide compound, and the use of a lower amount of peroxide compound with higher efficacy than a peroxide compound alone.

In some embodiments, the weight ratio of the peroxide compound to the zinc compound is from 1:8 to 4:1, optionally from 1:5 to 3:1, optionally from 1:3 to 2:1, optionally from 1:2 to 1:1, further optionally about 1:1.

In some embodiments, the zinc compound is selected from zinc oxide, zinc citrate, zinc chloride, zinc acetate, zinc lactate, zinc salicylate, zinc sulfate, zinc phosphate, zinc tartrate and zinc nitrate. In certain embodiments, the zinc compound is zinc oxide. Zinc oxide and other zinc compounds have been previously found to be effective agents for inhibiting the growth of bacteria, including those prevalent in the oral cavity. However, as discussed in the Examples below, this antibacterial activity cannot explain the increase in whitening efficacy observed for the zinc compound-containing compositions.

In some embodiments, the zinc compound is present in a concentration of from 0.1 to 5 weight %, from 0.25 to 3 weight %, from 0.5 to 2 weight %, from 0.75 to 1.5 weight %, from 0.8 to 1.2 weight %, from 0.9 to 1.1 weight %, or about 1 weight %, based on the weight of the composition.

In some embodiments, the peroxide compound is hydrogen peroxide, a peroxide of an alkali metal, a peroxide of an alkaline earth metal, an organic peroxy compound, or a peroxy acid. Examples of peroxides of alkali metals include lithium peroxide, sodium peroxide, and potassium peroxide. Examples of alkaline earth metal peroxides include magnesium peroxide, calcium peroxide, and barium peroxide. Examples of organic peroxy compounds include urea peroxide, glyceryl hydrogen peroxide, alkyl hydrogen peroxides, dialkyl peroxides, diacyl peroxides, peroxy esters, benzoyl peroxide and monoperoxyphthalate.

In some embodiments, the peroxide compound is hydrogen peroxide (H₂O₂). In certain embodiments, the hydrogen peroxide is present as a complex with a polymer (i.e. the hydrogen peroxide is present as a bound peroxide). In certain embodiments, the polymer is polyvinylpyrrolidone (PVP). In some embodiments, the complex comprises from 17 to 22 weight % hydrogen peroxide, based on the weight of the complex. In some embodiments, the complex comprises about 18 weight % hydrogen peroxide, based on the weight of the complex.

In some embodiments, the peroxide compound is present in a concentration of from 0.005 to 3 weight %, from 0.01 to 1 weight %, from from 0.01 to 0.5 weight %, from 0.02 to 0.4 weight %, from 0.03 to 0.3 weight %, from 0.04 to 0.2 weight %, from 0.05 to 0.1 weight %, from 0.075 to 0.1 weight %, or from 0.09 to 0.1 weight %, based on the total weight of the composition.

In some embodiments, the composition is anhydrous. By the composition being “anhydrous”, it is meant that the composition contains 2 weight % or less free water. In other embodiments, the maximum amount of water is 1.5 weight %, 1 weight %, or 0.5 weight %. In certain embodiments, the composition contains no free water. When calculating the water content, water molecules that are part of a hydrate of a material are not counted.

In some embodiments, the composition is a toothpaste, a liquid, a gel, a whitening strip, or a composition which is applied to the teeth using a dental tray. In certain embodiments, the composition is a gel. In some embodiments, the gel is adapted to be applied to the teeth by brushing (e.g. in a similar way to toothpaste).

The present invention provides a method of increasing the whitening efficacy of an oral care composition which comprises a peroxide compound, the method comprising formulating the composition so as to further comprise a zinc compound, wherein the weight ratio of the peroxide compound to the zinc compound in the composition is from 1:10 to 5:1. The oral care composition may be as described in any of the above embodiments of the composition.

The present invention provides the use, in an oral care composition which comprises a peroxide compound, of a zinc compound to increase the whitening efficacy of the composition, wherein the weight ratio of the peroxide compound to the zinc compound in the composition is from 1:10 to 5:1. The oral care composition may be as described in any of the above embodiments of the composition.

In some embodiments of the above method and use, the weight ratio of the peroxide compound to the zinc compound is from 1:8 to 4:1, optionally from 1:5 to 3:1, optionally from 1:3 to 2:1, optionally from 1:2 to 1:1, further optionally about 1:1.

In some embodiments of the above method and use, the zinc compound is selected from zinc oxide, zinc citrate, zinc chloride, zinc acetate, zinc lactate, zinc salicylate, zinc sulfate, zinc phosphate, zinc tartrate and zinc nitrate. In certain embodiments, the zinc compound is zinc oxide. Zinc oxide and other zinc compounds have been previously found to be effective agents for inhibiting the growth of bacteria, including those prevalent in the oral cavity. However, as discussed in the Examples below, this antibacterial activity cannot explain the increase in whitening efficacy observed for the zinc compound-containing compositions.

In some embodiments of the above method and use, the zinc compound is present in the composition in a concentration of from 0.1 to 5 weight %, from 0.25 to 3 weight %, from 0.5 to 2 weight %, from 0.75 to 1.5 weight %, from 0.8 to 1.2 weight %, from 0.9 to 1.1 weight %, or about 1 weight %, based on the weight of the composition.

In some embodiments of the above method and use, the peroxide compound is hydrogen peroxide, a peroxide of an alkali metal, a peroxide of an alkaline earth metal, an organic peroxy compound, or a peroxy acid. Examples of peroxides of alkali metals include lithium peroxide, sodium peroxide, and potassium peroxide. Examples of alkaline earth metal peroxides include magnesium peroxide, calcium peroxide, and barium peroxide. Examples of organic peroxy compounds include urea peroxide, glyceryl hydrogen peroxide, alkyl hydrogen peroxides, dialkyl peroxides, diacyl peroxides, peroxy esters, benzoyl peroxide and monoperoxyphthalate.

In some embodiments of the above method and use, the peroxide compound is hydrogen peroxide (H₂O₂). In certain embodiments, the hydrogen peroxide is present as a complex with a polymer (i.e. the hydrogen peroxide is present as a bound peroxide). In certain embodiments, the polymer is polyvinylpyrrolidone (PVP). In some embodiments, the complex comprises from 17 to 22 weight % hydrogen peroxide, based on the weight of the complex. In some embodiments, the complex comprises about 18 weight % hydrogen peroxide, based on the weight of the complex.

In some embodiments of the above method and use, the peroxide compound is present in the composition in a concentration of from 0.005 to 3 weight %, from 0.01 to 1 weight %, from from 0.01 to 0.5 weight %, from 0.02 to 0.4 weight %, from 0.03 to 0.3 weight %, from 0.04 to 0.2 weight %, from 0.05 to 0.1 weight %, from 0.075 to 0.1 weight %, or from 0.09 to 0.1 weight %, based on the total weight of the composition.

In some embodiments of the above method and use, the composition is anhydrous. By the composition being “anhydrous”, it is meant that the composition contains 2 weight % or less free water. In other embodiments, the maximum amount of water is 1.5 weight %, 1 weight %, or 0.5 weight %. In certain embodiments, the composition contains no free water. When calculating the water content, water molecules that are part of a hydrate of a material are not counted.

In some embodiments, the composition is a toothpaste, a liquid, a gel, a whitening strip, or a composition which is applied to the teeth using a dental tray. In certain embodiments, the composition is a gel. In some embodiments, the gel is adapted to be applied to the teeth by brushing (e.g. in a similar way to toothpaste).

In any of the above embodiments of the oral care composition, method and use, the compositions may further comprise additional ingredients. These additional ingredients may include, but are not limited to, diluents, silicones (such as dimethicone and DC8-7016 silicone fluid), bicarbonate salts, surfactants, sweeteners, flavorants, pigments, anticalculus or tartar control agents, polymers (such as xanthan gum, carboxymethylcellulose, carrageenan gum, polyvinylpyrrolidone) and mixtures thereof.

In some embodiments, the oral care compositions of the present invention comprise at least one bicarbonate salt useful for example to impart a “clean feel” to teeth and gums due to effervescence and release of carbon dioxide. The one or more additional bicarbonate salts are optionally present in a total amount of about 0.1 wt. % to about 50 wt. %, for example about 1 wt. % to 20 wt. %, by total weight of the composition.

The oral care compositions of the invention may also comprise at least one surfactant. Any orally acceptable surfactant, most of which are anionic, nonionic or amphoteric, can be used. One or more surfactants are optionally present in a total amount of about 0.01 wt. % to about 10 wt. %, for example, from about 0.05 wt. % to about 5 wt. %, or from about 0.1 wt. % to about 2 wt. % by total weight of the composition.

The oral care compositions of the present invention may comprise at least one sweetener (such as, for example, sodium saccharin), useful for example to enhance taste of the composition. One or more sweeteners are optionally present in a total amount depending strongly on the particular sweetener(s) selected, but typically 0.005 wt. % to 5 wt. %, by total weight of the composition, optionally 0.005 wt. % to 1 wt. %, further optionally 0.05 wt. % to 0.5 wt. % by total weight of the composition.

The compositions of the present invention may also comprise at least one flavorant, useful for example to enhance taste of the composition. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 1 wt. % by total weight of the composition.

The compositions of the invention may comprise at least one colorant. Colorants herein include pigments, coated dyes, protected dyes, lakes and agents imparting a particular luster or reflectivity such as pearling agents. Any orally acceptable colorant can be used. One or more colorants are optionally present in a total amount of from about 0.001 wt. % to about 20 wt. %, for example, from about 0.01 wt. % to about 10 wt. %, or from about 0.1 wt. % to about 5 wt. %, by total weight of the composition.

The compositions of the present invention may comprise a saliva stimulating agent useful, for example, in amelioration of dry mouth. One or more saliva stimulating agents are optionally present in saliva stimulating effective total amount.

The compositions of the present invention may include antisensitivity agents. Such agents may be added in effective amounts, e.g., from about 1 wt. % to about 20 wt. % by weight based on the total weight of the composition, depending on the agent chosen.

### EXAMPLES

### Example 1

Two gel compositions were formulated, as shown in Table 1. Both the Comparative and the Example compositions contained 0.55 weight % of a PVP-H₂O₂ complex (wherein the concentration of H₂O₂ in the complex was 18 weight %, based on the weight of the complex). The concentration of hydrogen peroxide in each of these compositions was therefore 0.1 weight %, based on the weight of the composition. These compositions differed only in that the Example composition also contained 1 weight % zinc oxide, whereas the Comparative composition contained no zinc oxide (with the Comparative composition containing an additional 1 weight % of one of the non-silicone polymers, in order to make the total weight of the composition up to 100 weight %).

TABLE 1

| | Weight % | |
|---|---|---|
| | Comparative | Example |
| Material | composition | composition |
| Silicones | 45.55 | 45.55 |
| Non-silicone polymers | 53.00 | 52.00 |
| Sweetener and flavor | 0.90 | 0.90 |
| PVP complexed with H₂O₂ | 0.55 | 0.55 |
| Zinc oxide | 0.00 | 1.00 |
| Total | 100.00 | 100.00 |

### Example 2

The whitening efficacy of the Example and Comparative compositions from Table 1, above, was measured in a laboratory test using stained bovine incisors. The incisors were first prepared by brushing with a solution of a peroxide-free toothpaste (which contained no other tooth bleaching agents) for 30 minutes. The L* value of the incisors was measured using a Spectroshade spectrophotometer and software. Only those incisors having a L* value of from 60 to 68 were selected for the study. The incisors were then sorted into groups of four, so that the incisors in a particular group had the same L* value.

Each group of four teeth (mounted so that only the surface of the teeth was exposed) was then subjected to six treatment cycles, with each cycle being two treatment applications (as described below) to mimic twice-daily usage. A first group of four teeth was treated using the Comparative composition from Table 1, above, and a second group of four teeth was treated using the Example composition from Table 1, above. Each treatment application had the following four steps:
(1) Approximately 50 mg of the composition under test was spread evenly across the four teeth.
(2) The teeth were inverted and submerged in a trough containing pH 7.4 aqueous phosphate buffer flowing at a rate of 2 ml/min, for 15 minutes.
(3) The teeth were removed from the trough and any remaining composition was gently removed using a laboratory wipe.
(4) The teeth were inverted and submerged in a trough containing pH 7.4 aqueous phosphate buffer for 15 minutes to rehydrate.

The Spectroshade spectrometer and software were used to measure the L*a*b* values of the teeth which were then used to calculate ΔW*, which is a common whitening index used for tooth whitening studies. The values shown in Table 2 below are the average of ΔW* as measured for each of the four individual teeth in the group. As discussed above, each cycle corresponds to two treatment applications.

TABLE 2
whitening efficacy

| | ΔW* | | | | | | |
|---|---|---|---|---|---|---|---|
| | baseline | 1 cycle | 2 cycles | 3 cycles | 4 cycles | 5 cycles | 6 cycles |
| Comparative | 0 | −0.25 | −0.33 | −0.41 | −0.52 | −0.70 | −0.79 |
| composition | | | | | | | |
| Example | 0 | −0.64 | −0.96 | −1.16 | −1.29 | −1.48 | −1.62 |
| composition | | | | | | | |

The evaluation of the color of the stained teeth was quantified using measurement of the L*a*b* color space, using a Spectroshade spectrometer and software. L*a*b* refers to stain score in accordance with the Commission International de L'Eclairage Laboratory (CIELAB) color scale, with L* being lightness-darkness scale, a* being red-green chroma, and b* being yellow-blue chroma. From measurement of the L*a*b* values, the whitening index ΔW* was calculated, wherein:

ΔW*=W*final−W*initial

and

W*=(a*²+b*²+(L*−100)²)^{1/2}.

The more negative the value of ΔW*, the closer the tooth color is to white.

As can be seen from Table 2, above, the whiteness improvement of teeth treated with the Example composition (which contained zinc oxide) was greater than twice that of the teeth treated with the Comparative composition, after 1, 2, 3, 4, 5 and 6 cycles of treatment.

This was a surprising result, particularly given that the increase in whitening efficacy of the Example composition cannot be explained by any antibacterial activity of the zinc oxide (as no bacteria were introduced into either the Example or the Comparative compositions).

### Example 3

In accordance with the procedures explained in Example 2, the whitening index used for tooth whitening studies (ΔW*) were applied to teeth which were independently exposed to both a 1:1 ratio of Hydrogen Peroxide: Zinc (1% Hydrogen Peroxide Gel), as well as a 4.5:1 ratio of Hydrogen Peroxide: Zinc (4.5% Hydrogen Peroxide Gel). Wherein the percent weight of hydrogen peroxide in the gel represents the weight relative to the total composition.

1:1 ratio of HP:Zinc (1% HP gel)

TABLE 3

| | Formula Weight % | |
|---|---|---|
| | Comparative | Example |
| Material | composition | composition |
| DC8-7016 Silicone Fluid | 30.00 | 30.00 |
| Dimethicone 350 CST | 14.00 | 13.00 |
| Plasticized Hydrocarbon Base | 30.10 | 30.10 |
| Sodium Saccharin | 0.30 | 0.30 |
| Flavor | 0.60 | 0.60 |
| PVP complexed with hydrogen peroxide | 5.50 | 5.50 |
| PVP | 19.5 | 19.5 |
| Zinc oxide | — | 1.00 |

TABLE 4
Whitening efficacy

| | ΔW | | | | | | |
|---|---|---|---|---|---|---|---|
| Group | Baseline | 2 cycles | 4 cycles | 6 cycles | 8 cycles | 10 cycles | 12 cycles |
| Comparative | 0 | −2.17 | −3.05 | −3.59 | −3.89 | −4.09 | −4.75 |
| composition | | | | | | | |
| Example | 0 | −2.42 | −3.43 | −3.88 | −4.14 | −4.57 | −5.41 |
| Composition | | | | | | | |
| % Imp | N/A | 11.5% | 12.5% | 8.1% | 6.4% | 11.7% | 13.9% |

4.5:1 ratio of HP:Zinc (4.5% HP gel)

TABLE 5

| | Formula Weight % | |
|---|---|---|
| | Comparative | Example |
| Material | composition | composition |
| DC8-7016 Silicone Fluid | 30.00 | 30.00 |
| Dimethicone 350 CST | 12.00 | 13.00 |
| Plasticized Hydrocarbon Base | 30.10 | 30.10 |
| Sodium Saccharin | 0.30 | 0.30 |
| Flavor | 0.60 | 0.60 |
| PVP complexed with hydrogen peroxide | 25.00 | 25.00 |
| Zinc oxide | — | 1.00 |

TABLE 6
Whitening efficacy

| | ΔW | | | | | | |
|---|---|---|---|---|---|---|---|
| Group | Baseline | 2 cycles | 4 cycles | 6 cycles | 8 cycles | 10 cycles | 12 cycles |
| Comparative | 0 | −4.28 | −6.19 | −7.02 | −7.89 | −8.50 | −9.25 |
| Composition | | | | | | | |
| Example | 0 | −5.04 | −6.85 | −8.00 | −9.13 | −10.07 | −11.01 |
| Composition | | | | | | | |
| % Imp | N/A | 10.7% | 10.7% | 14.0% | 15.7% | 18.5% | 19.0% |

Similar to the results demonstrated in Example 2, these figures represent surprising results. And the increase in whitening efficacy of the Example composition cannot be explained by the antibacterial activity of the zinc oxide (as no bacteria were introduced into either the Example or the Comparative compositions).

### Example 3

An experiment was conducted to determine whether or not the zinc oxide present in the Example composition affected the pH of saliva. For each of the Example and Comparative compositions in Table 1, above, a slurry was prepared containing 10 weight % of the composition dispersed in artificial saliva solution. Three aliquots of each solution were taken after 10, 20 and 30 minutes of agitation. The aliquots were centrifuged and the pH of the supernatant was measured. The results are shown in Table 7, below (average of the pH measured for the three aliquots).

TABLE 7
Impact on pH

| | pH of supernatant | | | |
|---|---|---|---|---|
| | baseline | 10 minutes | 20 minutes | 30 minutes |
| Comparative | 6.71 | 6.91 | 6.98 | 7.11 |
| composition | | | | |
| Example | 6.71 | 6.84 | 6.94 | 7.56 |
| composition | | | | |

As can be seen from Table 7, above, no significant difference in pH was observed between the Example composition and the Comparative composition within the treatment time as used for each cycle of the whitening experiment in Example 2, above. The lack of any significant pH difference between the Example and Comparative compositions at all time periods during the whitening cycle makes the increased whitening efficacy of the Example composition particularly surprising, as this effect cannot be attributed to any difference in pH between the two compositions. Without being bound by any theory, it is hypothesized that the increased whitening efficacy observed for the zinc oxide-containing composition might be due to the zinc oxide stabilizing the reactive oxygen species of hydrogen peroxide for oxidation.

What is claimed is:

1. An oral care composition comprising a peroxide compound, wherein the peroxide compound is present in a concentration of from 0.01 to 0.5 weight % based on the total weight of the composition, and a zinc compound, wherein the weight ratio of the peroxide compound to the zinc compound is from 1:10 to 5:1, and wherein the composition is anhydrous, meaning that the composition contains 2 weight % or less free water.

2. The oral care composition of claim 1, wherein the weight ratio of the peroxide compound to the zinc compound is from 1:8 to 4:1.

3. The oral care composition of claim 2, wherein the weight ratio of the peroxide compound to the zinc compound is from 1:5 to 3:1.

4. The oral care composition of claim 3, wherein the weight ratio of the peroxide compound to the zinc compound is from 1:3 to 2:1.

5. The oral care composition of claim 4, wherein the weight ratio of the peroxide compound to the zinc compound is 1:1.

6. The oral care composition of claim 1, wherein the zinc compound is selected from zinc oxide, zinc citrate, zinc chloride, zinc acetate, zinc lactate, zinc salicylate, zinc sulphate, zinc phosphate, zinc tartrate and zinc nitrate.

7. The oral care composition of claim 6, wherein the zinc compound is present in a concentration of from 0.1 to 5 weight %, optionally from 0.75 to 1.5 weight %, based on the weight of the composition.

8. The oral care composition of claim 1, wherein the peroxide compound is hydrogen peroxide present as a complex with_polyvinylpyrrolidone, a peroxide of an alkali metal, a peroxide of an alkaline earth metal, an organic peroxy compound, or a peroxy acid.

9. The oral care composition of claim 8, wherein the peroxide compound is present in a concentration of from 0.05 to 0.1 weight %, based on the total weight of the composition.

10. The oral care composition of claim 1, wherein the composition is a dentifrice, wherein the dentifrice is toothpaste, gel, or a whitening strip.
